SEQUENCE LISTING (25 sequences)

SEQ ID NO: 1 - protein, 10 aa, Artificial Sequence
  synthetic
EQKLISEEDL

SEQ ID NO: 2 - protein, 11 aa, Artificial Sequence
  synthetic
YPYDVPDYASL

SEQ ID NO: 3 - protein, 7 aa, Artificial Sequence
  synthetic
DYKDDDK

SEQ ID NO: 4 - DNA, 57 bp, Artificial Sequence
  synthetic
cgagtgtacaagaaaatgcctagttccggaatcgaatctgatgtttaagatcttcca

SEQ ID NO: 5 - DNA, 56 bp, Artificial Sequence
  synthetic
ggaagatcttaaacatcagattcgattccggaactaggcattttcttgtacactcg

SEQ ID NO: 6 - DNA, 6 bp, Artificial Sequence
  synthetic
tccgga

SEQ ID NO: 7 - DNA, 69 bp, Artificial Sequence
  synthetic
ccggtgaacaaaaactcatctcagaagaggatctgggggaacaaaaactcatctcagaagaggatctgt

SEQ ID NO: 8 - DNA, 69 bp, Artificial Sequence
  synthetic
ccggacagatcctcttctgagatgagtttttgttcccccagatcctcttctgagatgagtttttgttca

SEQ ID NO: 9 - protein, 21 aa, Artificial Sequence
  synthetic
EQKLISEEDLGEQKLISEEDL

SEQ ID NO: 10 - DNA, 6 bp, Artificial Sequence
  synthetic
tccggt

SEQ ID NO: 11 - DNA, 6 bp, Artificial Sequence
  synthetic
tccgga

SEQ ID NO: 12 - DNA, 48 bp, Artificial Sequence
  synthetic
ggctctgcgcatttctccagctctagactggagtccgaggtatgacgc

SEQ ID NO: 13 - DNA, 48 bp, Artificial Sequence
  synthetic
gcgtcatacctcggactccagtctagagctggagaaatgcgcagagcc

SEQ ID NO: 14 - DNA, 6 bp, Artificial Sequence
  synthetic
tctaga

SEQ ID NO: 15 - DNA, 63 bp, Artificial Sequence
  synthetic
ctagttatccttatgacgtccctgattatgccggatatccttatgacgtccctgattatgcct

SEQ ID NO: 16 - DNA, 63 bp, Artificial Sequence
  synthetic
ctagaggcataatcagggacgtcataaggatatccggcataatcagggacgtcataaggataa

SEQ ID NO: 17 - protein, 19 aa, Artificial Sequence
  synthetic
YPYDVPDYAGYPYDVPDYA

SEQ ID NO: 18 - DNA, 6 bp, Artificial Sequence
  synthetic
tctagt

SEQ ID NO: 19 - DNA, 6 bp, Artificial Sequence
  synthetic
tctaga

SEQ ID NO: 20 - DNA, 36 bp, Artificial Sequence
  synthetic
cagcgccggcgcagctccggactggagccacccgac

SEQ ID NO: 21 - DNA, 36 bp, Artificial Sequence
  synthetic
gtcgggtggctccagtccggagctgcgccggcgctg

SEQ ID NO: 22 - DNA, 5 bp, Artificial Sequence
  synthetic
ccgga

SEQ ID NO: 23 - DNA, 5 bp, Artificial Sequence
  synthetic
ccggt

SEQ ID NO: 24 - DNA, 5 bp, Artificial Sequence
  synthetic
ctaga

SEQ ID NO: 25 - DNA, 5 bp, Artificial Sequence
  synthetic
ctagt

(19) United States
(12) Patent Application Publication
(10) Pub. No.: US 2004/0241684 A1
(43) Pub. Date: Dec. 2, 2004

(54) Directed protein modification by iterative sequence insertion

(76) Inventors: Michael Nathan Nitabach, New York, NY (US); Todd Clayton Holmes, New York, NY (US)

Correspondence Address: KLAUBER & JACKSON, 411 HACKENSACK AVENUE, HACKENSACK, NJ, 07601

(21) Appl. No.: 10/483,538

(22) PCT Filed: Jul. 10, 2002
(86) PCT No.: PCT/US02/22252

Related U.S. Application Data
(60) Provisional application No. 60/304,957, filed on Jul. 12, 2001.

(51) Int. Cl. C12Q 1/68; C12P 19/34

(52) U.S. Cl. 435/6; 435/91.2

(57) ABSTRACT

A facile method is provided for the iterative insertion of two or more preselected polynucleotide sequences into another preselected polynucleotide sequence, particularly where the preselected sequences encode one or more of the same or different peptides, and a protein, respectively, such that the properties of the protein are modified to include that of the peptides. By iteratively adding copies of the peptides, the number of insertion iterations necessary to achieve the desired modified properties of the protein is controlled and minimal.

### FIELD OF THE INVENTION

This invention relates to the field of protein engineering, and particularly, methods of preparation and production of modified polynucleotides and e.g.chimeric proteins. Methods are provided for preparing modified polynucleotides containing iterative, tandem insertion of at least one first preselected polynucleotide sequence into a second preselected polynucleotide sequence wherein the preselected sequences encode one or more of the same or different peptides, and a protein, respectively, such that the properties of the protein are modified to include that of the peptides. The invention also relates to gene fragments useful for achieving such iterative insertion for production of the chimeric protein. Moreover, the invention also provides for lots useful for the iterative, tandem insertion of at least one first preselected polynucleotide sequence into a second preselected polynucleotide sequence.

### BACKGROUND OF THE INVENTION

Expression of recombinant proteins in bacteria, cultured eukaryotic cells, and transgenic organisms is a widely-used technique in basic biological research, drug development, and biotechnology. Moreover, such proteins are often modified versions of naturally-occuring proteins, the modifications provided for any of a number of purposes, such as enhanced detectability in the case of proteins for which isolation, quantitation or localization are desirable, and improved biophysical or biochemical properties, in the case of protein reagents for experimental or therapeutic purposes. In the case of isolation or detectability, immunological methods are frequently employed to detect and/or purify the expressed recombinant protein. This can be done by using antibodies or specific receptors that are able to specifically recognize the particular protein expressed, or by inserting into the protein a directly detectable moiety, such as a fluorescent polypeptide sequence. In the example of a therapeutically-useful protein, even if for in vitro use, the properties of the protein may be altered, for example, by chemical modification or cross-linking to another moiety to provide altered properties of the conjugate.

For detectability, the foregoing method has the disadvantage that it only works for proteins against which antibodies have been raised or a known receptor exists. When one is working with a newly isolated protein gene-product, no antibodies or receptors specific for that protein are likely to exist. In order to raise an antibody or identify a receptor, much time and effort are needed, and the results can be quite variable. Sometimes, an effective antibody can never be generated. Furthermore, antibodies that recognize epitopes that are inherent to both native and expressed recombinant proteins cannot distinguish between the native and expressed proteins. In many cases, it is desirable to detect or purify only the expressed recombinant protein, and not the related native protein.

In order to avoid these problems, the well-known technique of epitope tagging has been devised. Because the protein of interest is being expressed from a recombinant expression vector comprising a cDNA encoding the protein, as well as other DNA elements that drive expression, allow for the bacterial replication of the vector, and allow for the selection of cells that have incorporated the vector, etc., a small DNA segment that encodes an already-characterized small epitope can be easily inserted into the protein coding sequence of the recombinant expression vector. If this DNA segment is inserted into the recombinant expression vector in the correct position and reading frame, then the protein expressed will contain both the entire amino acid sequence of the protein of interest, as well as the small epitope.

This technique has the advantage that a variety of small epitopes exist for which well-characterized highly specific immunological reagents exist that can be easily purchased. Examples of such widely used epitopes include the “c-Myc” and “HA” epitopes, which have the amino acid sequences, EQKLISEEDL (SEQ ID NO.1) and YPYDVPDYASL (SEQ ID NO.2), respectively. Polyclonal and monoclonal antibodies that are highly specific for these epitopes can be purchased from any of a large number of suppliers, and can be used for immunoprecipitation, purification, Western blotting, immunocytochemical detection, etc., of recombinant proteins that contain the respective epitope tags. These uses, and others, are well known to practitioners in the biological arts.

In the case of altering the biophysical or biochemical properties of proteins, insertion of amino acid sequences is performed in similar fashion as that of an epitope tag. Receptor-binding sequences, hydrophobic domains, hydrophilic domains, and other known modifications have been carried out to alter the properties of a protein.

While it is well known in the biological arts that epitope tagging as well as other sequence insertions into a protein is a widely applicable and useful technique, as currently applied this technique has certain limitations. First, depending on the particular enzymatic techniques used to insert the DNA fragment encoding the sequence into the cDNA, it may be difficult to select the particular position within the protein coding region that the sequence is inserted. This can be important for a number of reasons: It may be necessary to insert the sequence only in a particular region within the recombinant protein to avoid interfering with the function of the protein, such as its enzymatic activity, its ability to form multimers, etc. Also, in the case of an epitope tag, the ability of the epitope-specific immunological reagents to bind to the epitope may depend on the exact location of the epitope insertion—some sites of insertion may be sterically hindered from antibody-epitope binding. This is also applicable to any other sequence to be inserted.

Second, the biological properties of the protein into which the sequence is inserted may depend heavily on the number of copies of the sequence that are inserted into the recombinant protein. This is applicable in the instance where the efficacy of immunological purification and/or detection is dependent on the number of inserted sequences, or the altered biophysical or biochemical properties dependent on the number of inserted sequences. This can occur in cases of recombinant proteins expressed at very low levels, or where the three-dimensional structure of the recombinant protein partially or completely prevents access of the receptor or binding partner to the inserted sequences.

In the instance of a therapeutically-useful protein, the aforementioned chemical modifications may not be specific, and in the case of cross-linking, a heterogeneous population of products may be obtained and the desired modified protein a minority therein. Purification methods are then necessary to isolate the desired conjugate.

In both of the foregoing examples, it may not be known a priori how many of the desirably inserted sequences are necessary to achieve the enhanced properties desired, yet the minimal amount of modification is desired to simultaneously add the desired new characteristic yet leave the original protein intact such that it may exhibit its natural properties.

It is towards the development and practice of a facile method for the stepwise insertion into a protein sequence of from one to a plurality of a preselected amino acid sequence that the present invention is directed.

The citation of any reference herein should not be deemed as an admission that such reference is available as prior art to the instant invention.

### SUMMARY OF THE INVENTION

In accordance with the present invention, a method is provided for preparing a modified polynucleotide, comprising an iterative, tandemly inserted plurality of at least one first preselected polynucleotide sequence into a second preselected polynucleotide sequence by carrying out at least the steps of:

(a) providing a plasmid vector comprising the second preselected polynucleotide sequence;

(b) inserting a restriction site at a desired site in the second preselected polynucleotide sequence, the restriction site not preexisting in the plasmid vector;

(c) treating the plasmid vector with a restriction endonuclease specific for the restriction site to cleave the plasmid vector at the restriction site, producing a cleaved plasmid vector;

(d) inserting a synthetic gene fragment comprising the first preselected polynucleotide sequence into the cleaved plasmid vector to form a modified plasmid vector, the synthetic gene fragment comprising at one end a sequence which when inserted into the cleaved plasmid vector at the restriction site reconstitutes the restriction site, and having at its other end a sequence which when inserted into the cleaved plasmid vector at the restriction site does not reconstitute the restriction site;

(e) isolating and amplifying the plasmid vector and selecting a modified plasmid vector that includes the first preselected polynucleotide sequence inserted into the second preselected polynucleotide sequence; and

(f) repeating steps (c) to (e) for each iteration to tandemly insert an additional first preselected polynucleotide sequence in the second preselected polynucleotide sequence in the modified plasmid vector.

By way of non-limiting examples, the inserting a restriction site in the second preselected polynucleotide sequence may be carried out by using mutagenic oligonucleotide primers that hybridize to the desired location of the insertion and that contain the desired restriction enzyme cleavage site, or it may be carried out by using a polymerase chain reaction with primers modified to contain the desired restriction site. The synthetic gene fragment that includes the first preselected polynucleotide sequence has overhangs that are compatible with overhangs produced by the cleavage of the plasmid vector with the restriction endonuclease, and moreover, at one (5′ or 3′) end, has a sequence in the adjacent double-stranded region such that upon ligation with the overhanging end created by cleavage of the restriction site reforms the restriction site; and at the other (3′ or 5′) end a sequence in the adjacent double-stranded region such that upon ligation with the overhanging end created by cleavage of said restriction site does not reform the restriction site.

Upon the first insertion step of a first polynucleotide sequence, and the subsequent insertion steps of one or more additional first polynucleotide sequences upon iteration of the insertion steps, the plasmid vector is referred to as a modified plasmid vector. Moreover, the same or different first polynucleotide sequences may be inserted in subsequent insertion steps, such that the iterative insertions may be tandem copies of the same polynucleotide sequence, or a pattern of alternating first polynucleotide sequences, or an entirely dissimilar tandem series of first polynucleotide sequences.

In one embodiment, the inserted polynucleotide sequences may be recognition sites for polynucleotide binding proteins, such as regulators, promoters, etc. In a preferred embodiment, the first polynucleotide sequence encodes for one or two, or possibly more, tandem copies of a peptide, of tandem copies of two different peptides, and the second polynucleotide sequence is a protein into which two or more copies of the peptide or peptides are desirably inserted, for example, to impart new and desirable properties to the protein. The protein may be any protein. The peptide may be, by way of non-limiting example, one, two, or more copies of an epitope tag, a fluorescent peptide, a hydrophilic peptide, a hydrophobic peptide, a sequence recognition site, a receptor recognition site, or a combination of any of the foregoing. Other inserted sequences are embraced herein. Each iteration of the insertion process described above may insert a different first polynucleotide sequence, provided that the overhanging ends and adjacent sequences of each sequence used are the same, as described above, for a particular application, such that the restriction site is reconstructed at only one end of each new first polynucleotide sequence insertion.

Preferably, the first preselected polynucleotide encodes a peptide and the second preselected polynucleotide sequence encodes a protein.

It is an object of the invention to provide for a method for iteratively introducing additional copies of at least one first polynucleotide sequence tandemly within the sequence of second polynucleotide sequence, wherein preferably, a particular number of inserted sequences impart a desirable characteristic to the second polynucleotide sequence, and such insertion steps may be easily repeated until the second polynucleotide sequence possesses the desired characteristics. Preferably, the first polynucleotide sequence encodes at least one peptide and the second polynucleotide sequence encodes a protein, and a certain number of tandem repeats of the peptide within the protein imparts a desirable characteristic. Such desirable characteristics include but are not limited to facile detectability of the protein by inserted tandem repeats of an epitope tag or fluorescent peptide, or in the case of altered protein structure of function, the addition of a targeting region, or altered hydrophobicity or hydrophilicity.

Each repeat of the insertion steps can be used to add a different or the same peptide, or alternating repeats of two or more different peptides. Various patterns of insertion are embraced herein.

Thus, a method is provided for producing a chimeric protein comprising at least two copies of at least one preselected peptide sequence tandemly inserted into a desired site in a preselected protein sequence, wherein said chimeric protein has properties of both the at least one preselected peptide and the preselected protein, comprising carrying out aforementioned method with an additional step following step (e) of evaluating the chimeric protein for the desired properties, and repeating steps (c) through (e) until said chimeric protein has the desired properties.

In a particular embodiment, a method is provided for producing a chimeric protein comprising at least two tandem repeats of at least one preselected peptide within a preselected protein comprising the steps of:

(a) providing a plasmid vector comprising a preselected polynucleotide sequence encoding the protein;

(b) inserting a restriction site at a desired site in the preselected polynucleotide sequence encoding the protein, said restriction site not preexisting in the plasmid vector;

(c) treating said plasmid vector with a restriction endonuclease specific for the restriction site to cleave the plasmid vector at the restriction site, producing a cleaved plasmid vector;

(d) inserting a synthetic gene fragment comprising a polynucleotide sequence encoding the peptide into the cleaved plasmid vector, the synthetic gene fragment comprising at one end a sequence which when inserted into the cleaved plasmid vector at the restriction site reconstitutes the restriction site, and having at its other end a sequence which when inserted into the cleaved plasmid vector at the restriction site does not reconstitute the restriction site;

(e) isolating and amplifying the plasmid vector and selecting the plasmid vector that expresses a chimeric protein comprising the peptide inserted into the protein; and

(f) repeating steps (c) to (e) to tandemly insert the peptide in said protein to produce the chimeric protein.

In a preferred but non-limiting embodiment, the inserting a restriction site in said polynucleotide sequence encoding the protein may be carried out by using mutagenic oligonucleotide primers that hybridize to the desired location of the insertion and that contain the desired restriction enzyme cleavage site, or by using a polymerase chain reaction with primers modified to contain the desired restriction site. The synthetic gene fragment comprising the polynucleotide sequence encoding said peptide has overhangs compatible with overhangs produced by cleavage of the plasmid vector with the restriction endonuclease, and moreover, at one (5′ or 3′) end, has a sequence in the adjacent double-stranded region such that upon ligation with the overhanging end created by cleavage of said restriction site reforms said restriction site; and at the other (3′ or 5′) end a sequence in the adjacent double-stranded region such that upon ligation with the overhanging end created by cleavage of said restriction site does not reform the restriction site.

Preferably, the peptide comprises one or more copies of an epitope tag, a fluorescent peptide, a hydrophilic peptide, a hydrophobic peptide, a sequence recognition site, or a receptor recognition site. Alternatively, each iteration can insert a synthetic gene fragment encoding a different peptide, or a pattern of additions can be inserted. While each synthetic gene fragment may have a different encoded peptide, each will have the same termini as described above, to maintain the pattern of reconstructing the restriction site at only one end of each newly inserted synthetic gene fragment.

In a further embodiment, a method is provided for producing a chimeric protein comprising at least two copies of a preselected peptide sequence tandemly inserted into a desired site in a preselected protein sequence, wherein the chimeric protein has properties of both the preselected peptide and the preselected protein, comprising carrying out the foregoing method with an additional step following step (e) of evaluating the chimeric protein for the properties, and repeating steps (c) through (e) until the chimeric protein has the desired properties.

The present invention is also directed to a kit for the iterative, tandem insertion of a first preselected polynucleotide sequence into a second preselected polynucleotide sequence comprising at least one 5′-phosphorylated synthetic gene fragment that comprises a first preselected polynucleotide sequence for insertion into a particular restriction site cleaved into identical overhanging ends with asymmetric reconstitution of the restriction site, the synthetic gene fragment having a 5′ end and a 3′ end, the synthetic gene fragment comprising:

(i) a preselected polynucleotide sequence encoding one or more peptides selected from the group consisting of an epitope tag, a fluorescent peptide, a hydrophilic peptide, a hydrophobic peptide, a sequence recognition site, and a receptor recognition site;

(ii) at the 5′ or 3′ end of the synthetic gene fragment a single-stranded overhang complementary to the overhanging ends of the cleaved restriction site and an adjacent double-stranded region with a sequence such that upon ligation with one of the overhanging ends of the cleaved restriction site reforms the restriction site; and

(iii) at the 3′ or 5′ end, respectively, of the synthetic gene fragment a single-stranded overhang complementary to the overhanging ends of the cleaved restriction site and an adjacent double-stranded region with a nucleotide adjacent to the single-stranded overhang that abolishes recognition of the restriction site by a restriction endonuclease that recognizes the restriction site;

instructions for the use of the kit, and one or more of the following optional components:

(a) a restriction enzyme that recognizes and specifically cleaves the particular restriction site;

(b) alkaline phosphatase enzyme;

(c) T4 DNA ligase enzyme;

(d) the components required to introduce a restriction site into a desired site within the second preselected polynucleotide sequence;

(e) transformation-competent E. coli; or

(f) monoclonal and/or polyclonal antibodies specific for the epitope encoded by the first preselected polynucleotide sequence.

The present invention is also directed to synthetic gene fragments that comprise a preselected polynucleotide sequence for insertion into a cleaved restriction site and asymmetric reconstitution thereof, as described in the foregoing methods. The synthetic gene fragments contain a preselected polynucleotide sequence; at one end (5′ or 3′) a single-stranded overhang with sequence complementary to the overhang created upon cleavage of the restriction site, and with a sequence in the adjacent double-stranded region such that upon ligation with the overhanging end created by cleavage of said restriction site reforms said restriction site; and at the other (3′ or 5′) end a single-stranded overhang with sequence complementary to the overhang created upon cleavage of said restriction site, and with a sequence in the adjacent double-stranded region such that upon ligation with the overhanging end created by cleavage of said restriction site does not reform the restriction site. End of the fragment that does not reconstitute the restriction site has a nucleotide adjacent to the single-stranded overhang such that upon ligation to the cohesive ends formed by cleavage of the restriction site, forms a sequence that is not recognizable by the restriction endonuclease.

As noted above, to carry out the method of the invention, a restriction site is introduced into the plasmid vector which does not preexist. Thus, the synthetic gene fragments for asymmetric reconstitution match the selected restriction site. However, the asymmetric gene fragments of the invention may be used for other purposes and are not so restricted to the aforementioned use. By way of non limiting examples, if the restriction site is Bsp E1, the synthetic gene fragment at one end is 5′-CCGGA-3′ (SEQ ID NO.22), which reconstitutes the restriction site, and the sequence at the other end is 5′-CCGGT-3′ (SEQ ID NO.23), which does not reconstitute the restriction site. In another example, the restriction site is an Xba I site, said sequence at one end is 5′-CTAGA-3′ (SEQ D NO.24) and said sequence at said other end is 5′-CTAGT-3′ (SEQ ID NO.25).

Preferably, the preselected polynucleotide sequence in the synthetic gene fragment encodes a peptide, such as, but not limited to, one or more copies of an epitope tag, a fluorescent peptide, a hydrophilic peptide, a hydrophobic peptide, a sequence recognition site, or a receptor recognition site.

Thus, it is an object of the invention to provide a method for preparing a modified polynucleotide comprising an iterative, tandemly inserted plurality of first preselected polynucleotide sequences into a second preselected polynucleotide sequence comprising the steps of:

(a) providing a plasmid vector comprising said second preselected polynucleotide sequence;

(b) inserting a restriction site at a desired site in said second preselected polynucleotide sequence, said restriction site not preexisting in said plasmid vector;

(c) treating said plasmid vector with a restriction endonuclease specific for said restriction site to cleave said plasmid vector at said restriction site, producing a cleaved plasmid vector;

(d) inserting a synthetic gene fragment comprising a first preselected polynucleotide sequence into said cleaved plasmid vector to form a modified plasmid vector, said synthetic gene fragment comprising at one end a sequence which when inserted into said cleaved plasmid vector at said restriction site reconstitutes said restriction site, and having at its other end a sequence which when inserted into said cleaved plasmid vector at said restriction site does not reconstitute said restriction site;

(e) isolating and amplifying said plasmid vector and selecting a modified plasmid vector comprising said first preselected polynucleotide sequence inserted into said second preselected polynucleotide sequence; and

(f) repeating steps (c) to (e) at least once using a same or different first polynucleotide sequence to additionally tandemly insert said same or different first preselected polynucleotide sequence in said second preselected polynucleotide sequence of said modified plasmid vector. The invention is also directed to a polynucleotide sequence prepared by the aforementioned method.

In the foregoing method, providing a restriction site in said second preselected polynucleotide sequence may be carried out by using mutagenic oligonucleotide primers that hybridize to the desired location of the insertion and that contain the desired restriction enzyme cleavage site, or by using a polymerase chain reaction with primers modified to contain the desired restriction site.

In the foregoing method, the synthetic gene fragment comprising said first preselected polynucleotide sequence may have overhangs compatible with overhangs produced by cleavage of said plasmid vector with said restriction endonuclease.

In the foregoing method, the second preselected polynucleotide sequence may encode a protein.

In the foregoing method, the first preselected polynucleotide sequence may encode a peptide.

In the foregoing method where the first preselected polynucleotide is a peptide, the peptide may comprise one or more copies of an epitope tag, a fluorescent peptide, a hydrophilic peptide, a hydrophobic peptide, a sequence recognition site, or a receptor recognition site.

In the foregoing method, the first preselected polynucleotide may encode a peptide and said second preselected polynucleotide sequence may encode a protein.

It is further object of the invention to provide a method for producing a chimeric protein comprising a plurality of preselected peptide sequences tandemly inserted into a desired site in a preselected protein sequence, wherein said chimeric protein has properties of both said preselected peptide sequences and said preselected protein, comprising carrying out the aforementioned method with an additional step following step (e) of evaluating said chimeric protein for said properties, and repeating steps (c) through (e) until said chimeric protein has said properties. The peptide may comprise one or more copies of one or more epitope tags. The properties may comprise native biological activity of said preselected protein and immunodetectability of said one or more epitope tags.

It is yet a further object of the present invention to provide a method for producing a chimeric protein comprising at least two tandem repeats of one or more preselected peptides within a preselected protein comprising the steps of:

(a) providing a plasmid vector comprising a preselected polynucleotide sequence encoding said protein;

(b) inserting a restriction site at a desired site in said preselected polynucleotide sequence encoding said protein, said restriction site not preexisting in said plasmid vector;

(c) treating said plasmid vector with a restriction endonuclease specific for said restriction site to cleave said plasmid vector at said restriction site, producing a cleaved plasmid vector;

(d) inserting a synthetic gene fragment comprising a polynucleotide sequence encoding one of said one or more preselected peptides into said cleaved plasmid vector to form a modified plasmid vector, said synthetic gene fragment comprising at one end a sequence which when inserted into said cleaved plasmid vector at said restriction site reconstitutes said restriction site, and having at its other end a sequence which when inserted into said cleaved plasmid vector at said restriction site does not reconstitute said restriction site;

(e) isolating and amplifying said plasmid vector and selecting said modified plasmid vector that expresses a chimeric protein comprising said preselected peptide inserted into said protein; and

(f) repeating steps (c) to (e) at least once using a polynucleotide sequence encoding a same or different preselected peptide to additionally tandemly insert said same or different peptide in said chimeric protein to produce a further-modified chimeric protein.

Inserting a restriction site in said polynucleotide sequence encoding said protein may be carried out by using mutagenic oligonucleotide primers that hybridize to the desired location of the insertion and that contain the desired restriction enzyme cleavage site, or by using a polymerase chain reaction with primers modified to contain the desired restriction site. The synthetic gene fragment comprising said polynucleotide sequence encoding said peptide may have overhangs compatible with overhangs produced by cleavage of said plasmid vector with said restriction endonuclease. The peptide may comprise one or more copies of an epitope tag, a fluorescent peptide, a hydrophilic peptide, a hydrophobic peptide, a sequence recognition site, or a receptor recognition site.

It is still yet a further object of the invention to provide a method for producing a chimeric protein comprising at least two copies of one or more preselected peptide sequences tandemly inserted into a desired site in a preselected protein sequence, wherein said chimeric protein has properties of both said one or more preselected peptides and said preselected protein, comprising carrying out the aforementioned method with an additional step following step (e) of evaluating said chimeric protein for said properties, and repeating steps (c) through (e) until said chimeric protein has said properties.

It is another object of the invention to provide a chimeric protein prepared by any of the aforementioned methods.

It is furthermore an object of the present invention to provide a synthetic gene fragment comprising a preselected polynucleotide sequence for insertion into a restriction site cleaved into identical overhanging ends and asymmetric reconstitution thereof, said synthetic gene fragment having a first end and a second end, said synthetic gene fragment comprising:

(a) a preselected polynucleotide sequence;

(b) at said first end of said synthetic gene fragment a single-stranded overhang complementary to said overhanging ends of said cleaved restriction site and an adjacent double-stranded region with a sequence such that upon ligation with one of said overhanging ends of said cleaved restriction site reforms said restriction site; and

(c) at said second end of said synthetic gene fragment a single-stranded overhang complementary to said overhanging ends of said cleaved restriction site and an adjacent double-stranded region with a sequence such that upon ligation with one of said overhanging ends of said cleaved restriction site does not reform said restriction site.

In the aforementioned synthetic gene fragment, the first end may be 5′ and said second end may be 3′; or, the first end may be 3′ and said second end may be 5′. The single-stranded overhang at said second end complementary to said overhanging ends of said cleaved restriction site that upon ligation with one of said overhanging ends of said cleaved restriction site will not reform said restriction site has a nucleotide adjacent to said single-stranded overhang that abolishes recognition of said restriction site by a restriction endonuclease that recognizes said restriction site. Examples include, for a restriction site being a Bsp EI site, said sequence at said first end is 5′-CCGGA-3′ (SEQ ID NO.22) and said sequence at said second end is 5′-CCGGT-3′ (SEQ ID NO.23). For a restriction site being Xba I site, said sequence at said first end is 5′-CTAGA-3′ (SEQ ID NO.24) and said sequence at said second end is 5′-CTAGT-3′ (SEQ ID NO.25). In the aforementioned synthetic gene fragment, the preselected polynucleotide sequence may encode a peptide, such as one or more copies of an epitope tag, a fluorescent peptide, a hydrophilic peptide, a hydrophobic peptide, a sequence recognition site, or a receptor recognition site. Moreover, the method described above may be used in the preparation of any synthetic gene fragment.

It is still a further object of the invention to provide a kit for the iterative, tandem insertion of at least one first preselected polynucleotide sequence into a second preselected polynucleotide sequence, said kit comprising at least one 5′-phosphorylated synthetic gene fragment that comprises a first preselected polynucleotide sequence for insertion into a particular restriction site, cleaved into identical overhanging ends and asymmetric reconstitution thereof, the synthetic gene fragment having a 5′ end and a 3′ end, the synthetic gene fragment comprising:

(i) a preselected polynucleotide sequence encoding one or more peptides selected from the group consisting of an epitope tag, a fluorescent peptide, a hydrophilic peptide, a hydrophobic peptide, a sequence recognition site, and a receptor recognition site;

(ii) at the 5′ or 3′ end of the synthetic gene fragment a single-stranded overhang complementary to the overhanging ends of the cleaved restriction site and an adjacent double-stranded region with a sequence such that upon ligation with one of the overhanging ends of the cleaved restriction site reforms the restriction site; and

(iii) at the 3′ or 5′ end, respectively, of the synthetic gene fragment a single-stranded overhang complementary to the overhanging ends of the cleaved restriction site and an adjacent double-stranded region with a nucleotide adjacent to the single-stranded overhang that abolishes recognition of the restriction site by a restriction endonuclease that recognizes the restriction site;

instructions for the use of the kit, and one or more of the following optional components:

(a) a restriction enzyme that recognizes and specifically cleaves the particular restriction site;

(b) alkaline phosphatase enzyme;

(c) T4 DNA ligase enzyme;

(d) the components required to introduce a restriction site into a desired site within the second preselected polynucleotide sequence;

(e) transformation-competent E. coli; or (f) monoclonal and/or polyclonal antibodies specific for the epitope encoded by the first preselected polynucleotide sequence.

Moreover, the kit described herein may be useful in preparation of chimeric proteins.

These and other aspects of the present invention will be better appreciated by reference to the following Detailed Description.

### DETAILED DESCRIPTION OF THE INVENTION

The present invention relates to the modification of recombinant proteins expressed in bacteria, cultured eukaryotic cells, or transgenic organisms. More specifically, the present invention relates to a facile method for modifying the properties of a protein to a preselected extent by iteratively inserting tandemly into the sequence of the recombinant protein two or more copies of the same or different preselected amino acid sequences, such that the number of insertion iterations necessary to achieve the preselected extent is controlled and minimal. In another related aspect, a method for the iterative tandem insertion of a plurality of the same or different preselected amino acid sequences into a protein is provided by the steps of creating a new restriction endonuclease site in the DNA encoding the desired protein, and then inserting and ligating at the site an asymmetric double-stranded oligonucleotide with cohesive single-stranded overhanging ends comprising a preselected DNA sequence encoding the inserted amino acid sequence, the oligonucleotide which reconstructs the restriction site only at one end of the inserted sequence. By repeated use of the asymmetric oligonucleotide (referred to herein interchangeably as a synthetic gene fragment), the subsequent iteration steps result in only a single new restriction site at one end of the inserted sequence, whereupon the next iteration adds only a single additional insert. By use of the asymmetric oligonucleotide and iterative insertions, one sequence at a time may be inserted into the recombinant protein. The properties imparted to the protein by each insertion may be determined, and the iterations stopped when the protein has the desired preselected properties.

As mentioned above, in the instance of epitope tagging, a preferred but non-limiting embodiment, a variety of small epitopes exist for which corresponding, well-characterized and highly-specific immunological reagents exist that can be easily purchased. Examples of such widely used epitopes include the “c-Myc” and “HA” epitopes, which have the amino acid sequences, EQKLISEEDL (SEQ ID NO.1) and YPYDVPDYASL (SEQ ID NO.2), respectively. Polyclonal and monoclonal antibodies that are highly specific for these epitopes can be purchased from any of a large number of suppliers, and can be used for immunoprecipitation, purification, Western blotting, immunocytochemical detection, etc., of recombinant proteins that contain the respective epitope tags. These uses, and others, are well known to practitioners in the biological arts.

It should be noted that while the foregoing discussion and following examples pertain to the facile, repetitive insertion of a peptide sequence into a protein, that the invention is not so limiting and extends to the iterative insertion of one polynucleotide sequence into another polynucleotide sequence. While either of the sequences may encode an amino acid sequence as discussed above, the method may be used simply for the production of a polynucleotide sequence with two or more tandemly-inserted polynucleotide sequences. Uses include evaluation of polynucleotides with sequences that interact with proteins, such as transcription factors and other nucleic acid sequence-specific binding proteins, and the aspect of the invention in which the sequences are expressed as proteins is not limiting.

To provide an example of prior art methods, consider the recombinant human beta-tubulin protein to be expressed in cultured insect cells. If no easily-obtainable antibody that specifically recognizes beta-tubulin is available, the recombinant expression vector containing the cDNA for human beta-tubulin can be modified enzymatically to insert in-frame at the 3′-end of the human beta-tubulin coding sequence a 30 base-pair DNA segment that encodes the c-Myc epitope. When this modified expression vector is transfected or otherwise introduced into cultured insect cells, the expressed recombinant protein will contain not only the human beta-tubulin polypeptide, but also the EQKLISEEDL (SEQ ID NO.1) c-Myc epitope peptide fused to its C-terminal end. This protein can then be purified and/or detected by using commercially-available anti-c-Myc-epitope antibodies, and does not require the generation of antibodies specific for human beta-tubulin. This allows for the immunological detection of a protein—e.g., one that is newly discovered-for which no specific antibodies already exist on a time scale that is much shorter than the time required to generate such specific antibodies.

While it is well known in the biological arts that epitope tagging is a widely applicable and useful technique, as currently applied this technique has certain limitations. As noted in the Background section, above, first, depending on the particular enzymatic techniques used to insert the DNA fragment encoding the epitope into the cDNA, it may be difficult to select the particular position within the protein coding region that the epitope is inserted. This can be important for a number of reasons: It may be necessary to insert the epitope only in a particular region within the recombinant protein to avoid interfering with the function of the protein, such as its enzymatic activity, its ability to form multimers, etc. Also, the ability of the epitope-specific immunological reagents to bind to the epitope may depend on the exact location of the epitope insertion-some sites of insertion may be sterically hindered from antibody-epitope binding.

Second, the efficacy of immunological purification and/or detection may depend heavily on the number of copies of the epitope tag that are inserted into the recombinant protein. This can occur in cases of recombinant proteins expressed at very low levels, or where the three-dimensional structure of the recombinant protein partially or completely prevents access of the anti-epitope tag antibodies to the inserted epitopes.

Two or more copies of the same or different first polynucleotide sequence may be inserted tandemly into the second polynucleotide sequence. For example, if A represents a polynucleotide sequence which may encode a peptide, such as an epitope tag, a fluorescent peptide or a hydrophobic peptide, the method may be carried out to insert A, then AA, then AAA, then AAAA into the second or recipient polynucleotide.

In another embodiment, A may represent two similar or dissimilar epitope tags in tandem and these may be inserted iteratively.

In yet another embodiment, let A, B and C be different first polynucleotides which may encode different peptides desirably inserted into a second polynucleotide. The method of the invention may be used to create the series: A, AB, ABA, ABAB, etc.; or A, AB, ABC, ABCA, ABCAB, etc. Depending on the selection of the end of the synthetic gene fragment that reconstructs the restriction site, the pattern of the foregoing additions could be A, BA, ABA, BABA, etc.; or A, BA, CBA, ACBA, BACBA, etc. The patterns of iterative insertions of the same or dissimilar first polynucleotides is not limiting and the invention embraces all such variations. The iterations are performed until the chimeric polynucleotide or polypeptide achieves the desired properties.

Besides epitope tagging, numerous other applications exist for the modification of recombinant proteins for which the same considerations and problems as described above for epitope tagging also exist. For example, insertion of fluorescent peptides or amino acid sequences is desirable to make a protein autofluorescent for localization or other detection methods. In the case of therapeutically-useful proteins or those for in vitro or in vivo experimental use, modification of the protein may be desirable to alter the biophysical, biochemical, or pharmacokinetic properties of the protein, i.e., to alter pK, affinity for a particular binding site or receptor, altered enzymatic activity, oral absorption, in-vivo metabolism, distribution in an animal, and/or excretion. In one non-limiting example, the hydrophilicity or hydrophobicity of a protein may be desirably altered to change its partitioning in vivo or in vitro, in order to target the protein to a certain site or alter its pharmacokinetics.

In all of the instances and uses above, new properties are desirably added to a preselected protein without negating its original or naturally-occurring properties, such that the modified protein has additional properties. As noted above, it is desirable to sequentially insert into a protein a particular preselected sequence, and then evaluate the protein for achievement of the desired additional property. If the first insertion does not achieve a satisfactory result, the insertion may be repeated to provide two copies of the preselected sequence in the protein. Again, evaluation of the modified protein will indicate whether the desired properties have been achieved or another round of preselected sequence insertion is indicated. This may be repeated until the desired properties are obtained.

However, a facile means for step-wise tandem insertion of a preselected amino acid sequence is not readily available. In order to avoid these problems with prior art methods of protein modification using the procedures routinely used for sequence insertion, the inventors herein have developed a method for the controlled, iterative tandem insertion into a protein sequence of a preselected amino acid sequence. The essence of the present invention is a method, reagents, and kit for the iterative insertion of any desired number of multiple copies of a preselected sequence into any desired location within a recombinant protein of interest.

In order to carry out the method of the invention, first a restriction site that does not already exist anywhere in the plasmid vector containing the coding region of the cDNA for the protein of interest may be introduced into the coding region using, by way of non-limiting example, the method described in U.S. Pat. Nos. 5,789,166 and 5,932,419. This introduced restriction site is referred to herein as a “novel restriction site.” This method allows for the introduction of a restriction enzyme cleavage site wherever it is desired to insert the preselected sequence, by designing mutagenic oligonucleotide primers that hybridize to the desired location of the insertion and that contain the desired restriction enzyme cleavage site. It should be noted that any method may be utilized to introduce the restriction site in the desired position, including but not limited to the use of a polymerase chain reaction with primers modified to contain the desired restriction site.

In a subsequent step, a synthetic gene fragment coding for one copy of the preselected amino acid sequence is provided that has overhangs that are compatible with the overhangs produced by cleavage of the novel restriction site introduced into the cDNA by any of a variety of methods as mentioned above, that has 5′ phosphate groups, and that will encode the preselected sequence in-frame with the cDNA coding region when inserted into the introduced novel restriction site. The preselected amino acid sequence may be a single copy of the sequence desirably inserted one at a time, or may be a tandem repeat, where each insertion of the preselected amino acid sequence adds two copies of the sequence desirably inserted. An essential feature of the invention is that the synthetic gene fragment comprising the coding sequence for the preselected amino acid sequence is designed so that the base pair (or pairs) adjacent to the overhang at either the 5′ or 3′ end, but not both, do not regenerate the novel restriction site when ligated into the digested cDNA, while the base pair (or pairs) adjacent to the other end of the fragment are chosen so as to regenerate the novel restriction site. Thus, the synthetic gene fragment comprising the coding sequence for the preselected amino acid sequence is asymmetric with regard to reconstructing the novel restriction site. As will be explained in more detail below, this novel aspect of the invention allows for the iterative tandem insertion of any desired number of copies of the preselected sequence.

As used herein, the preselected amino acid sequence refers to the sequence of amino acids inserted during each iteration of the process of the invention. If the preselected sequence is a single epitope tag, then the first insertion process inserts a single copy, and each successive iteration produces a recombinant product with 2, 3, 4, 5, etc. copies of the epitope tag tandemly inserted. If the preselected amino acid sequence is a tandem repeat of, for example, an epitope tag, then the first insertion process inserts the tandem repeat resulting in two copies of the exemplary epitope tag, and each successive insertion step would result in 4, 6, 8, 10, etc. copies of the exemplary epitope tag. It is within the teachings herein in the iteration process to use a combination of preselected sequences, such as the first insert being a tandem repeat, and then the successive insertions to be single copies. This modification of the procedure allows for an initial more coarse series of insertions and then, when the modified protein beings to approach the desired preselected properties, for a series of single insertions.

The plasmid containing the cDNA is then digested with the appropriate restriction enzyme to cleave the introduced novel restriction site, and the linearized plasmid is treated with either calf intestinal alkaline phosphatase or shrimp alkaline phosphatase to remove 5′ phosphates from the overhangs resulting from cleavage. This plasmid is then ligated to the synthetic gene fragment using T4 DNA ligase enzyme, and the resulting products are transformed into bacteria.

Clones are isolated, amplified, and sequenced, and a clone that contains in the appropriate frame and orientation a single copy of the synthetic gene fragment is selected for further analysis. This analysis involves the expression and detection of the modified recombinant protein using whatever method is needed to identify the preselected property imparted to the protein by the insertion of the preselected sequence. In the case of an epitope tag, immunological reagents specific for the epitope tag for immunoprecipitation, purification, Western blotting, and/or immunohistochemistry. In the case of a modified biophysical, biochemical or pharmacokinetic property of the protein, this may be assessed by routine methods.

A novel advantage of the present invention becomes apparent if and when it is determined that the protein with a single insertion of the preselected sequence is not detectable with a desired level of sensitivity. At this point, the selected plasmid clone can again be digested with the appropriate restriction enzyme. Because the inserted synthetic gene fragment was designed to regenerate the novel restriction enzyme recognition site only at one of its ends, digestion linearizes the plasmid at this position and leaves the inserted synthetic gene fragment in place, rather than excising the inserted gene fragment. This linearized plasmid is then ligated to the synthetic gene fragment as above, and clones are isolated, amplified, and sequenced. This time, a clone is selected for further analysis that contains not only the previously inserted single copy of the synthetic gene fragment, but now a second copy. This process can be iterated to insert any desired number of copies of the synthetic gene fragment into the cDNA encoding the protein of interest, as determined by the desired level of performance.

Selection of the novel restriction site and the corresponding DNA sequence encoding the preselected amino acid sequence that has overhangs that are compatible with the overhangs produced by cleavage of the novel restriction site (and asymmetrically reconstructs the restriction site at one end when ligated) will be readily selected by one of skill in the art based on the inserted sequence and the presence of existing restriction endonuclease sites in the vector.

Any preselected amino acid sequence may be used to carry out the methods of the invention, and its selection will of course depend on the desired properties to be imparted to the protein. As mentioned above, one use is to insert the minimum number of tandem repeats of an epitope tag into a recombinant protein to make it recognizable by a binding partner to the epitope tag, such as but not limited to an antibody, such that the expressed modified recombinant protein has a detectable epitope tag and retains desired properties of the native protein. As noted above, non-limiting examples of epitope tags are the c-myc and HA sequences. Many others are known and equally adaptable to the methods herein.

Endogenously-fluorescent amino acid sequences may also be inserted into a protein using the method herein to create a modified protein with sufficient detectability for the purposes intended.

Moreover, modifications to alter the biophysical or biochemical properties of a protein may be carried out using the iterative process described herein, in order to provide the minimal number of sequence insertions to achieve the desired modified properties without altering the properties of the protein beyond its intended utility. For example, a short hydrophilic or hydrophobic amino acid sequence may be inserted using the methods herein to alter the properties of the protein. An example of such a hydrophilic sequence is DYKDDDK (SEQ ID NO.3) described by Shih et al., J. Cell Biol. 136:1037-45. Another example is a polypeptide spacer of any length that may be inserted using the present method into the “chain” region of an amino-terminal “ball-and-chain” structure of a rapidly-inactivating ion channel, in order to modify the rate and other properties of channel inactivation.

The asymmetric synthetic oligonucleotides (also referred to herein as synthetic gene fragments) of the invention are characterized by having overhangs which are complementary to the overhangs generated by cleavage of the novel newly-introduced restriction sites, but have, at one end, an adjacent nucleotide which abrogates recognition of the ligated site by the restriction endonuclease. Thus, asymmetric reconstitution of a restriction site refers to the aforementioned oligonucleotides, which reconstitute the restriction site at one end but fail to do so at the other. Such synthetic oligonucleotides or gene fragments are prepared synthetically, as they cannot be generated by restriction endonuclease cleavage.

Complementary single-stranded oligonucleotides that comprise a sense and antisense strand encoding a desired polypeptide sequence as well as ends that will form single-stranded cohesive overhangs after annealing are synthesized chemically using standard techniques and then annealed to one another using standard techniques so as to result in the desired double-stranded synthetic gene fragment with single-stranded overhangs. The selection of the 5′ end or the 3′ end as the reconstituting end, and the other as the non-reconstituting end is optional-either end may be chosen as reconstituting, so long as the other end does not reconstitute the restriction site.

The present invention is also directed to modified polynucleotides and chimeric proteins prepared by the methods described above.

A kit may be prepared in accordance with the teachings herein for the iterative, tandem insertion of a first preselected polynucleotide sequence into a second preselected polynucleotide sequence. Such a kit will include at least one or more 5′-phosphorylated synthetic gene fragments that comprise a first preselected polynucleotide sequence for insertion into a particular restriction site cleaved into identical overhanging ends and asymmetric reconstitution thereof, the synthetic gene fragment having a 5′ end and a 3′ end, the synthetic gene fragment comprising:

(i) a preselected polynucleotide sequence encoding one or more peptides selected from the group consisting of an epitope tag, a fluorescent peptide, a hydrophilic peptide, a hydrophobic peptide, a sequence recognition site, and a receptor recognition site;

(ii) at the 5′ or 3′ end of the synthetic gene fragment a single-stranded overhang complementary to the overhanging ends of the cleaved restriction site and an adjacent double-stranded region with a sequence such that upon ligation with one of the overhanging ends of the cleaved restriction site reforms the restriction site; and

(ii) at the 3′ or 5′ end, respectively, of the synthetic gene fragment a single-stranded overhang complementary to the overhanging ends of the cleaved restriction site and an adjacent double-stranded region with a nucleotide adjacent to the single-stranded overhang that abolishes recognition of the restriction site by a restriction endonuclease that recognizes the restriction site;

instructions for the use of the kit, and one or more of the following optional components:

(a) a restriction enzyme that recognizes and specifically cleaves the particular restriction site;

(b) alkaline phosphatase enzyme;

(c) T4 DNA ligase enzyme;

(d) the components required to introduce a restriction site into a desired site within the second preselected polynucleotide sequence;

(e) transformation-competent E. coli; or

(f) monoclonal and/or polyclonal antibodies specific for the epitope encoded by the first preselected polynucleotide sequence.

Moreover, the kit described herein may be useful in preparation of chimeric proteins.

The invention is also directed to synthetic gene fragment compositions which comprise three parts: a 5′ terminus, a 3′ terminus, and an intervening preselected polynucleotide sequence which may encode a peptide or protein. The sequences of the termini, referred to herein as a first end and a second end, are provided for insertion into a cleaved restriction site and asymmetric reconstitution of the restriction site after insertion. The synthetic gene fragments have identical overhanging ends, and at the first end adjacent to the overhang is a double-stranded region with a sequence such that upon ligation with one of the overhanging ends of the cleaved restriction site reforms the restriction site; and at the second end, adjacent to the overhang is a double-stranded region with a sequence such that upon ligation with one of the overhanging ends of the cleaved restriction site does not reform the restriction site. Thus, the synthetic gene fragment compositions are useful for the iterative, tandem insertion of a one preselected polynucleotide sequence into another. Either the first end is 5′ and said second end is 3′, or the first end is 3′ and said second end is 5′. The preselected polynucleotide sequence is selected for the particular application in which a polynucleotide or protein is desirably modified by the insertion of two or more copies of a same or different polynucleotide or peptide sequence, and may be, by way of non-limiting example, a protein-binding polynucleotide sequence or a peptide encoding a an epitope tag, a fluorescent peptide, a hydrophilic peptide, a hydrophobic peptide, a sequence recognition site, or a receptor recognition site. The overhangs and asymmetric adjacent sequences may be selected based on the particular restriction site into which the insertion and asymmetric reconstitution of the restriction site is desired, such as for the uses described herein. Non-limiting examples of asymmetric ends for a Bsp EI restriction site are 5′-CCGGA-3 (SEQ ID NO. 22) and said sequence at said second end is 5′-CCGGT-3′ (SEQ ID NO. 23); for Xba I site, are 5′-CTAGA-3′ (SEQ ID NO. 24) and 5′-CTAGT-3′ (SEQ ID NO. 25).

The aforementioned examples and uses of such synthetic gene fragments are merely exemplary and the present invention embraces all such constructs and uses thereof.

The following examples are presented in order to more fully illustrate the preferred embodiments of the invention. They should in no way be construed, however, as limiting the broad scope of the invention.

### EXAMPLE 1

### Tagging Rat NMDAR2A with Mvc Epitope

A supercoiled circular bacterial plasmid containing downstream of the mammalian cytomegalovirus promoter a cDNA encoding the rat NMDA receptor 2A protein, and containing no endogenous Bsp EI restriction enzyme sites, was subjected to the method disclosed in U.S. Pat. Nos. 5,789,166 and 5,932,419 using as oligonucleotide primers the following: 5′-GAGTGTACAAGAAAATGCCTAGTTCCGGAATCGAATCTGATGTTTAAGATCTTCC A-3′ (SEQ ID NO.4) and 5′-TGGAAGATCTTAAACATCAGATTCGATTCCGGAACTAGGCATTTTCTTGTACAC TCG-3′ (SEQ ID NO.5). This resulted in the insertion of a Bsp EI restriction enzyme cleavage site (TCCGGA [SEQ ID NO.6]) in-frame between the codons of the cDNA encoding amino acids 1459 and 1460 of rat NMDAR2A. It would also have been possible to use other standard methods, such as polymerase chain reaction, to insert the Bsp EI site at this position in the NMDAR2A cDNA.

This modified cDNA was then cleaved with Bsp EI restriction enzyme under standard conditions, thus yielding a linearized plasmid with Bsp EI overhanging ends. This linearized plasmid was treated with alkaline phosphatase enzyme under standard conditions to remove 5′ phosphate groups.

The linearized, dephosphorylated plasmid was ligated under standard conditions to a double-stranded DNA fragment generated by annealing the following two complementary chemically-synthesized single-stranded oligonucleotides: 5′-CCGGTGAACAAAAACTCATCTCAGAAGGATCTGGGGAACAAAACTCATCT CAGAAGAGGATCTGT-3′ (SEQ ID NO.7) and 5′-CCGGACAGATCCTCTTCTGAGATGAGTGATGTTCCCCCAGATCCTCTTCTGAGAT GAGTTTTGTTCA-3′ (SEQ ID NO.8). This double-stranded DNA fragment encodes the peptide “EQKLISEEDLGEQKUSEEDL” (SEQ ID NO.9); two tandem repeats of the myc epitope with a glycine linker amino acid) and contains 5′-CCGG-3′ single-stranded overhangs at each end. These overhangs are exactly complementary to the overhangs created by Bsp EI digestion of the modified NDMAR2A cDNA-containing plasmid, thus allowing for “cohesive-end” ligation.

It should be noted that the next base on the first listed strand neighboring the 5′-CCGG-3′ overhang is T, so when this end of the double-stranded DNA fragment is ligated to the cut Bsp EI site in the plasmid, the sequence 5′-TCCGGT-3′ (SEQ ID NO.10) is created, which is not a Bsp EI site. By contrast, the next base neighboring the 5′-CCGG-3′ overhang on the other strand is A, so when this end of the double-stranded DNA fragment is ligated to the cut Bsp EI site in the plasmid, the sequence 5′-TCCGGA-3′ (SEQ ID NO. 11) is created, which is a Bsp EI site.

Clones resulting from this ligation reaction were sequenced, and one in which a single copy of the double stranded DNA fragment had been inserted in the correct orientation was selected for further analysis. The correct orientation is defined as that in which the coding strand of the rat NMDAR2A cDNA is the same strand as the coding strand of the myc epitope tags in the double-stranded DNA fragment.

Cultured HEK 293 mammalian kidney cells were transfected with this cDNA using standard techniques, cells were lysed under standard conditions, and cell lysates were separated using PAGE. Western blots were prepared, reacted with anti-myc epitope tag monoclonal antibody obtained from cell-culture supernatant of the well-known 9E10 hybridoma line, and then developed using standard enhanced chemiluminescence. No immunoreactivity at the expected molecular weight for NMDAR2A was observed, nor was there any immunoreactivity observed in the lysates of the myc-tagged NMDAR2A transfected cells that was not present in lysates of control untransfected cells. This indicated that we were unable to detect binding of the myc epitope-specific immunoreagents to the NMDAR2A recombinant protein that had been tagged with only 2 copies of the myc epitope.

The plasmid clone that contained a single copy of the “double myc” tag was cleaved with Bsp EI restriction enzyme under standard conditions. As explained above, a Bsp EI site was recreated only at the 3′-end of the inserted “double myc” DNA fragment, and not at the 5′-end. Thus, this Bsp EI cleavage resulted in a linearized plasmid that still contained the “double myc” DNA fragment at one end, and contained Bsp EI overhangs at both ends. This linearized plasmid was dephosphorylated using alkaline phosphatase under standard conditions. This linearized, dephosphorylated plasmid was then ligated under standard conditions to the “double myc” synthetic double-stranded DNA fragment. Clones resulting from this ligation reaction were sequenced, and one in which a single additional copy of the double stranded DNA fragment had been inserted in the correct orientation was selected for further analysis. The correct orientation is defined, as it was above, as that in which the coding strand of the rat NMDAR2A cDNA is the same strand as the coding strand of the myc epitope tags in the newly-inserted additional double-stranded DNA fragment. In other words, this clone now contained 4 copies of the myc epitope tag inserted in-frame and in tandem between amino acids 1459 and 1460 of rat NMDAR2A.

Cultured HEK 293 mammalian kidney cells were transfected with this cDNA containing 4 copies of the myc tag using standard techniques, cells were lysed under standard conditions, and cell lysates were separated using PAGE. Western blots were prepared, reacted with anti-myc epitope tag monoclonal antibody obtained from cell-culture supernatant of the well-known 9E10 hybridoma line, and then developed using standard enhanced chemiluminescence. Strong immunoreactivity at the expected molecular weight for NMDAR2A was observed, and this immunoreactivity observed in the lysates of the myc-tagged NMDAR2A transfected cells was not present in lysates of control untransfected cells. This indicated that we were now able to detect binding of the myc epitope-specific immunoreagents to the NMDAR2A recombinant protein that had been tagged with 4 copies of the myc epitope. In contrast, NMDAR2A recombinant protein tagged with only 2 copies of the myc epitope does not exhibit detectable binding to the myc-specific immunoreagents, and is thus not useful.

### EXAMPLE 2

### Tagging Rat NMDAR2D with HA Epitope

The same basic procedure was followed for the epitope tagging of Rat NMDAR2D recombinant protein in Example 1, above, but with the following differences. Instead of tagging with the myc epitope, NMDAR2D was tagged with the HA epitope. Instead of inserting the “double HA” DNA fragment into a Bsp EI site introduced into the NMDAR2D coding sequence, the DNA fragment was inserted into an Xba I site introduced into the NMDAR2D coding sequence. Finally, the “double HA” fragment insertion procedure needed to be iterated 3 times before the recombinant HA-tagged NMDAR2D protein could be detected with anti-HA monoclonal antibody (hybridoma line 12CA5) on a Western blot of lysates of transfected HEK 293 cells. This resulted in a recombinant protein with 6 total copies of the HA epitope. Note that while it is desirable to minimize the number of epitope tags inserted into the recombinant protein so as to reduce the chances of interfering with the inherent functional properties of the protein, it is not predictable a priori how many copies of the epitope will be needed to allow for robust binding of the tag-specific immunoreagents.

The oligonucleotides utilized for insertion of the Xba I site into NMDAR2D were as follows:

5′-
GGCTCTGCGCATTTCTCCAGCTCTAGACTGGAGTCCGAGGTATGACGC-
3′
(SEQ ID NO. 12)
and
5′-
GCGTCATACCTCGGACTCCAGTCTAGAGCTGGAGAAATGCGCAGAGCC-
3′.
(SEQ ID NO. 13)

This resulted in the insertion of an Xba I restriction enzyme cleavage site (TCTAGA [SEQ ID NO.14]) in-frame between the codons of the cDNA encoding amino acids 1318 and 1319 of rat NMDAR2D.

The “double HA” DNA fragment was generated by annealing the following two oligonucleotides: 5′-CTAGTTATCCTTATGACGTCCCTGATTATGCCGGATATCCTTATGACGTCCCTGA TTATGCCT-3′ (SEQ ID NO.15) and 5′-CTAGAGGCATAATCAGGGACGTCATAAGGATATCCGGCATAATCAGGGACGTC ATAAGGATAA-3′ (SEQ ID NO. 16). This double-stranded DNA fragment encodes the peptide “YPYDVPDYAGYPYDVPDYA” (SEQ ID NO.17); two tandem repeats of the HA epitope with a glycine linker amino acid) and contains 5′-CTAG-3′ single-stranded overhangs at each end. These overhangs are exactly complementary to the overhangs created by Xba I digestion of the Quickchange-modified NDMAR2D cDNA-containing plasmid, thus allowing for “cohesive-end” ligation.

It should be noted that the next base on the first listed strand neighboring the 5′-CTAG-3′ overhang is T, so when this end of the double-stranded DNA fragment is ligated to the cut Xba I site in the plasmid, the sequence 5′-TCTAGT-3′ (SEQ ID NO.18) is created, which is not an Xba I site. By contrast, the next base neighboring the 5′-CTAG-3′ overhang on the other strand is A, so when this end of the double-stranded DNA fragment is ligated to the cut Xba I site in the plasmid, the sequence 5′-TCTAGA-3′ (SEQ ID NO.19) is created, which is an Xba I site.

### EXAMPLE 3

### Tagging v-Src with Mvc Epitope

The same basic procedure was followed for the epitope tagging of v-Src recombinant protein described above, with the following differences. While the same Bsp EI “double myc” DNA fragment was used, and was ligated into a Bsp EI site introduced into the v-Src coding sequence, this site was introduced into the v-Src cDNA with the following two oligonucleotides: 5′-CAGCGCCGGCGCAGCTCCGGACTGGAGCCACCCGAC-3′ (SEQ ID NO.20) and 5′-GTCGGGTGGCTCCAGTCCGGAGCTGCGCCGGCGCTG-3′ (SEQ ID NO.21). This resulted in the insertion of a Bsp EI restriction enzyme cleavage site (TCCGGA [SEQ ID NO.11]) in-frame between the codons of the cDNA encoding amino acids 17 and 18 of v-Src. In addition, the “double myc” fragment insertion procedure needed to be iterated only once before the recombinant myc-tagged v-Src protein could be detected with anti-myc monoclonal antibody on a Western blot of lysates of transfected HEK 293 cells. In contrast to NMDAR2A and NMDAR2D, v-Src recombinant protein is detectable with only 2 copies of the myc epitope. This indicates that the disclosed procedure works not only with transmembrane proteins, such as NDMAR2A and NMDAR2D, but also with soluble proteins such as v-Src. The disclosed method and reagents may be used to epitope tag any recombinant protein, and is not limited to the particular examples disclosed herein.

Note, that under some circumstances it might be desirable to enzymatically cleave the epitope tags from the recombinant protein after purification. It is consistent with the present invention to include specific protease peptide cleavage sites in the epitope tag to enable this further manipulation of the epitope tagged recombinant protein.

While the invention has been described and illustrated herein by references to the specific embodiments, various specific material, procedures and examples, it is understood that the invention is not restricted to the particular material combinations of material, and procedures selected for that purpose. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

1. A method for preparing a modified polynucleotide, comprising an iterative, tandemly inserted plurality of first preselected polynucleotide sequences and a second preselected polynucleotide sequence, comprising the steps of:
(a) providing a plasmid vector comprising the second preselected polynucleotide sequence;
(b) inserting a restriction site at a desired site in the second preselected polynucleotide sequence, the restriction site not preexisting in the plasmid vector;
(c) treating the plasmid vector with a restriction endonuclease specific for the restriction site to cleave the plasmid vector at the restriction site, producing a cleaved plasmid vector;
(d) inserting a synthetic gene fragment comprising a first preselected polynucleotide sequence into the cleaved plasmid vector to form a modified plasmid vector, the synthetic gene fragment comprising at one end a sequence which, when inserted into the cleaved plasmid vector at the restriction site, reconstitutes the restriction site, and having at its other end a sequence which, when inserted into the cleaved plasmid vector at the restriction site, does not reconstitute the restriction site;
(e) isolating and amplifying the plasmid vector and selecting a modified plasmid vector comprising the first preselected polynucleotide sequence inserted into the second preselected polynucleotide sequence; and
(f) repeating steps (c) to (e) at least once using a same or different first polynucleotide sequence to additionally tandemly insert the same or different first preselected polynucleotide sequence in the second preselected polynucleotide sequence of the modified plasmid vector.

2. The method of claim 1, wherein step (b) is carried out by using mutagenic oligonucleotide primers that hybridize to the desired location of the insertion and that contain the desired restriction enzyme cleavage site, or by using a polymerase chain reaction with primers modified to contain the desired restriction site.

3. The method of claim 1, wherein the synthetic gene fragment comprising the first preselected polynucleotide sequence has overhangs compatible with overhangs produced by cleavage of the plasmid vector with the restriction endonuclease.

4. The method of claim 1, wherein the second preselected polynucleotide sequence encodes a protein.

5. The method of claim 1, wherein the first preselected polynucleotide sequence encodes a peptide.

6. The method of claim 5, wherein the peptide comprises one or more copies of an epitope tag, a fluorescent peptide, a hydrophilic peptide, a hydrophobic peptide, a sequence recognition site, or a receptor recognition site.

7. The method of claim 1, wherein the first preselected polynucleotide encodes a peptide and the second preselected polynucleotide sequence encodes a protein.

8. A method for producing a chimeric protein comprising at least two tandem repeats of one or more preselected peptides within a preselected protein comprising the steps of:
(a) providing a plasmid vector comprising a preselected polynucleotide sequence encoding the protein;
(b) inserting a restriction site at a desired site in the preselected polynucleotide sequence encoding the protein, the restriction site not preexisting in the plasmid vector;
(c) treating the plasmid vector with a restriction endonuclease specific for the restriction site to cleave the plasmid vector at the restriction site, producing a cleaved plasmid vector;
(d) inserting a synthetic gene fragment comprising a polynucleotide sequence encoding one of the one or more preselected peptides into the cleaved plasmid vector to form a modified plasmid vector, the synthetic gene fragment comprising at one end a sequence which, when inserted into the cleaved plasmid vector at the restriction site, reconstitutes the restriction site, and having at its other end a sequence which, when inserted into the cleaved plasmid vector at the restriction site, does not reconstitute the restriction site;
(e) isolating and amplifying the plasmid vector and selecting the modified plasmid vector that expresses a chimeric protein comprising the preselected peptide inserted into the protein;
(f) expressing the chimeric protein and evaluating the chimeric protein for the properties of both the one or more preselected peptides and the preselected protein; and
(g) repeating steps (c) to (f) at least once using a polynucleotide sequence encoding a same or different preselected peptide to additionally tandemly insert the same or different peptide in the chimeric protein to produce a further-modified chimeric protein.

9. The method of claim 8, further comprising expressing the chimeric protein and evaluating the chimeric protein for the properties of both the one or more preselected peptides and the preselected protein.

10. The method of claim 8, wherein the inserting a restriction site in the polynucleotide sequence encoding the protein is carried out by using mutagenic oligonucleotide primers that hybridize to the desired location of the insertion and that contain the desired restriction enzyme cleavage site, or by using a polymerase chain reaction with primers modified to contain the desired restriction site.

11. The method of claim 8, wherein the synthetic gene fragment comprising the polynucleotide sequence encoding the peptide has overhangs compatible with overhangs produced by cleavage of the plasmid vector with the restriction endonuclease.

12. The method of claim 8, wherein the peptide comprises one or more copies of an epitope tag, a fluorescent peptide, a hydrophilic peptide, a hydrophobic peptide, a sequence recognition site, or a receptor recognition site.

13. (canceled)

14. A synthetic gene fragment comprising a preselected polynucleotide sequence for insertion into a restriction site cleaved into identical overhanging ends and asymmetric reconstitution thereof, the synthetic gene fragment having a 5′ end and a 3′ end, the synthetic gene fragment comprising:
(a) a preselected polynucleotide sequence encoding one or more peptides selected from the group consisting of an epitope tag, a fluorescent peptide, a hydrophilic peptide, a hydrophobic peptide, a sequence recognition site, and a receptor recognition site;
(b) at said 5′ or 3′ end of the synthetic gene fragment a single-stranded overhang complementary to the overhanging ends of the cleaved restriction site and an adjacent double-stranded region with a sequence such that upon ligation with one of the overhanging ends of the cleaved restriction site reforms the restriction site; and
(c) at the 3′ or 5′ end, respectively, of the synthetic gene fragment a single-stranded overhang complementary to the overhanging ends of the cleaved restriction site and an adjacent double-stranded region with a nucleotide adjacent to the single-stranded overhang that abolishes recognition of the restriction site by a restriction endonuclease that recognizes the restriction site.

15. The synthetic gene fragment of claim 14, wherein the restriction site is a Bsp EI site, the sequence at the first end is 5′-CCGGA-3′ (SEQ ID NO.21) and the sequence at the second end is 5′-CCGGT-3′ (SEQ ID NO.22).

16. The synthetic gene fragment of claim 14, wherein the restriction site is an Xba I site, the sequence at the first end is 5′-CTAGA-3′ (SEQ ID NO.23) and the sequence at the second end is 5′-CTAGT-3′ (SEQ ID NO.24).

17. The synthetic gene fragment of claim 14, wherein the preselected polynucleotide sequence encodes a peptide.

18. The synthetic gene fragment of claim 14, wherein the peptide comprises one or more copies of an epitope tag, a fluorescent peptide, a hydrophilic peptide, a hydrophobic peptide, a sequence recognition site, or a receptor recognition site.

19. A kit for the iterative, tandem insertion of at least one first preselected polynucleotide sequence into a second preselected polynucleotide sequence, the kit comprising:
(a) at least one 5′-phosphorylated synthetic gene fragment comprising a first preselected polynucleotide sequence for insertion into a restriction site cleaved into identical overhanging ends and asymmetric reconstitution thereof, the synthetic gene fragment having a 5′ end and a 3′ end, the synthetic gene fragment comprising:
(i) a preselected polynucleotide sequence encoding one or more peptides selected from the group consisting of an epitope tag, a fluorescent peptide, a hydrophilic peptide, a hydrophobic peptide, a sequence recognition site, and a receptor recognition site;
(ii) at the 5′ or 3′ end of the synthetic gene fragment a single-stranded overhang complementary to the overhanging ends of the cleaved restriction site and an adjacent double-stranded region with a sequence such that upon ligation with one of the overhanging ends of the cleaved restriction site reforms the restriction site; and
(iii) at the 3′ or 5′ end, respectively, of the synthetic gene fragment a single-stranded overhang complementary to the overhanging ends of the cleaved restriction site and an adjacent double-stranded region with a nucleotide adjacent to the single-stranded overhang that abolishes recognition of the restriction site by a restriction endonuclease that recognizes the restriction site;
(b) a restriction enzyme that recognizes and specifically cleaves the particular restriction site;
(c) alkaline phasphatase enzyme;
(d) T4 DNA ligase enzyme;
(e) the compounds required to introduce a restriction site into a desired site within the second preselected polynucleotide sequence;
(f) transforrnation-competent E. coli; or
(g) monoclonal and/or polyclonal antibodies specific for the epitope encoded by the first preselected polynucleotide sequence; and
(h) instructions for the use of the kit.

20. (canceled)

21. (canceled)

22. (canceled)
